(19) United States
(12) Patent Application Publication
(10) Pub. No.: US 2003/0014123 A1
(43) Pub. Date: Jan. 16, 2003

(54) Cementless hip joint endoprosthesis as a surface replacement for the proximal femur

(76) Inventors: Peter Copf, Tubingen (DE); Werner Hein, Leipzig (DE)

Correspondence Address: LERNER AND GREENBERG, P.A., Post Office Box 2480, Hollywood, FL, 33022-2480, US

(21) Appl. No.: 10/151,045

(22) Filed: May 20, 2002

(30) Foreign Application Priority Data
May 18, 2001 (DE) 101 24 705.2
Jun. 23, 2001 (DE) 101 30 366.1

(51) Int. Cl. A61F 2/36

(52) U.S. Cl. 623/23.14; 623/23.29; 623/23.32

(57) ABSTRACT

A hip-joint endoprosthesis as a surface replacement for the proximal femur is proposed, which hip-joint endoprosthesis can be secured in the neck of the femur without cement.

### DESCRIPTION

The invention relates to a hip-joint endoprosthesis as a surface replacement for the proximal femur, with a cap, said cap having an outer face, which interacts with a socket, and an inner face, and with a stem, which stem can be connected to the cap.

These hip-joint endoprostheses are cemented into the femoral neck in the prior art. Using cement to secure the hip-joint endoprosthesis can cause a great many problems for the patient. For example, loosening of the hip-joint endoprosthesis in the femoral neck can occur as a result of the different temperature expansion coefficients of bone, cement and hip-joint endoprosthesis and as a result of the ageing of the cement. The stability of the connection between hip-joint endoprosthesis and femoral neck can also be reduced from the outset as a result of a change in the volume of the cement when it sets. Finally, the cement can also lead to rejection reactions. All of these potential problems, which can be occasioned by the use of cement, are known from the relevant specialist literature and will not be specifically detailed here.

The object of the invention is to make available a hip-joint endoprosthesis as a surface replacement for the proximal femur, which hip-joint endoprosthesis permits, without use of cement, reliable, load-bearing, permanent and compatible securing of the hip-joint endoprosthesis in the femoral neck.

In a hip-joint endoprosthesis with a cap, said cap having an outer face, which interacts with a socket, and an inner face, and with a stem, which stem can be connected to the cap, this object is achieved by the fact that the stem has a lattice-like structure.

Upon implantation of the hip-joint endoprosthesis according to the invention, the lattice-like structure is positioned in the area of the spongy substance of the femoral neck. This affords the possibility that, after implantation, the spongy substance, at least some of which has been removed in the area of the femoral neck, will grow into the lattice-like structure and thus create a connection between stem and femoral neck that can be subjected to high loads and is sufficiently elastic. A certain elasticity of the connection between hip-joint endoprosthesis and femoral neck is necessary in order to induce the spongy substance to grow and to regenerate throughout the lifetime of the hip-joint endoprosthesis. A secure and load-bearing fit of the hip-joint endoprosthesis in the femoral neck is thus guaranteed throughout the lifetime of the hip-joint endoprosthesis. In addition to the stated advantages of the invention, the hip-joint endoprosthesis according to the invention has all the advantages, known from the prior art, of a surface replacement for the ball of a joint. One of these advantages lies is that the femoral neck does not have to be removed, so that, at the end of the lifetime of the hip-joint endoprosthesis according to the invention, it is possible to implant a second hip-joint endoprosthesis in which the femoral neck is removed. In addition, the replacement of the surface of the head of the femur is much less traumatic than the use of a hip-joint endoprosthesis in which the femoral neck has to be removed.

According to the invention, provision can be made for the cap and stem, in the implanted state, to be connected to one another with a frictional fit or shape fit, or for the cap to rest on the stem in the implanted state.

In the hip-joint endoprosthesis according to the invention, the anchoring is effected almost exclusively via the spongy substance of the femoral neck. An introduction of force into the cortical substance of the femoral neck is provided for only to a very small degree since, because of the greater rigidity of the cortical substance, this introduction of force would lead to no forces or only very low forces being introduced into the spongy substance, and, as a consequence of this, stimulation of the growth of the spongy substance would not take place or would do so to an insufficient degree. This would lead in the long term to loosening of the hip-joint endoprosthesis in the femoral neck.

It is particularly advantageous if the rigidity of the stem, in particular in the longitudinal direction of the stem, is greater than the spongy substance surrounding the stem and/or if the rigidity of the stem, in particular in the longitudinal direction of the stem, is less than the cortical substance of the femoral neck. In this configuration of the stem, a sufficiently great force is introduced from the cap into the spongy substance via the stem in order to relieve the stress on the resection surfaces of the femoral head and to achieve a cushioning which is as close as possible to the natural cartilaginous tissue. In addition, the spongy substance is induced to grow by the introduction of force, without overstressing the spongy substance. From the literature (see Röhrle, H.; Scholten, W.; Grünert, A.: “Der Kraftfluss bei Hüftendoprothesen” in Arch. Orthop. Unfall-Cir. 89, 49-70, 1977), it is known that the modulus of elasticity of the spongy substance in the area in which the stem is anchored is approximately 1.21×10⁵ N/cm² and that for the cortical substance of the femoral neck it is approximately 1.45×10⁶ N/cm².

In further variants of the invention, the lattice-like structure of the stem is composed of openings or of rods and bridges and/or the lattice-like structure has thickened areas and/or depressions or a mesh-like structure so as to guarantee the best possible growth of the spongy substance into the lattice-like structure for the purposes of a secure and at the same time elastic connection between hip-joint endoprosthesis and femoral neck. In addition to an improved shape fit, the thickened areas and depressions increase the surface area of the stem, which has an advantageous effect in terms of the strength of the anchoring of the hip-joint endoprosthesis.

It has also proven expedient if the longitudinal axis of the stem extends in the direction of the greatest loading of the femoral neck, so that the stem can take up this load and introduce it, along its entire length or surface, into the spongy substance of the femoral neck.

The growth of the spongy substance into the endoprosthesis takes place in three phases. In the first phase, only small forces are to be transmitted from the endoprosthesis to the spongy substance (physiological force introduction). For this reason, a relative immobilization of the mutually contacting surfaces of endoprosthesis and spongy substance is necessary. In addition, the resection surfaces must match very precisely with the endoprosthesis. In this first phase, the stem is important for ensuring a good force distribution/force introduction. It relieves the stress on the conical bearing surface of the cap.

When, in the second phase, the conical bearing surface of the cap has connected to the spongy substance (bone formation and thus stiffening of the area around the cap), the force taken up by the stem decreases (shifting of the force flow on the cap).

If the spongy substance is permanently (third phase) unable to take up the forces which arise, the stem will permanently introduce some of these forces into the spongy substance. To ensure that the stem does not transmit all of the forces acting on the cap, it is advantageous if the stem is configured as follows: a) The first third of the stem should be connected to the cap in a flexurally rigid manner but, in the longitudinal direction, should as far as possible have a rigidity lying within the aforementioned limits. b) The second third of the stem should be flexible and, in the longitudinal direction, should have a rigidity lying within the aforementioned limits. c) The third third of the stem should be flexible and, in the longitudinal direction, should have a rigidity lying above the aforementioned limits.

To improve the connection between hip-joint endoprosthesis and femoral neck, provision can alternatively be made for the surface of the stem to be porous, periplasmal or of hydroxyapatite. If the surface is porous or periplasmal, the spongy substance can grow into the surface, which results in a correspondingly good interlocking and connection between spongy substance and hip-joint endoprosthesis. If the surface of the lattice-like structure or of the stem is of hydroxyapatite, the spongy substance can grow onto the surface, which also permits a secure and permanently load-beaving connection between hip-joint endoprosthesis and femoral neck.

To further improve the introduction of force into the femoral neck, the inner face of the cap corresponds to the surface of a double truncated cone because, in this configuration of the hip-joint endoprosthesis according to the invention, the resection surfaces can be produced easily and with the required precision by means of the rotary movement of a suitably configured tool. By a suitable choice of cone angle, it is possible to achieve the desired clamping action between the cap and the resection surfaces of the femoral neck bone. This clamping action is of special importance, in particular immediately after the operation, for the anchoring of the hip-joint endoprosthesis in the femoral neck. After the spongy substance has grown into the lattice-like structure, the significance of the contact surfaces between cap and bone being shaped as a double truncated cone is that they ensure an is as far as possible planar introduction of force into the femoral neck. By this means, the loads acting on the lattice-like structure, and on the spongy substance which has grown into this structure, are reduced to the required extent.

In order to ensure an optimum introduction of force from the hip-joint endoprosthesis into the femoral neck, the head of the femur is cut away until the cortical substance has been completely removed and anchoring of the hip-joint endoprosthesis in the area of the head of the femur is effected solely via the spongy substance and not via the cortical substance. However, care must be taken to ensure that the bearing capacity of the head of the femoral neck bone is not reduced too much.

To simplify the production of cap and stem, and to simplify the implantation, it has proven advantageous for the cap and the stem to be connected to one another by a clamp connection. This can be effected, for example, a conical recess in the stem and a corresponding peg on the cap. The cross section of the conical recess and of the peg can, for example, be round, polygonal, rectangular or square.

Finally, the outer face of the cap can be provided with a coating which lessens wear and/or reduces friction, so as to extend the lifetime of the hip-joint endoprosthesis according to the invention.

It has proven advantageous to produce the hip-joint endoprosthesis at least partially from titanium, in particular TiAl6V4, steel or a CoCr alloy, by casting and/or forging. The cap can be made of steel or of a CoCr alloy, inter alia because of their good tribology properties, while the stem can be made from TiAl6V4. The hip-joint endoprosthesis can optionally be made at least partially of sintered metal or porous ceramic or biodegradable material.

Further advantages and advantageous embodiments of the invention can be gathered from the attached drawings, from their description, and from the patent claims.

### DRAWING

In the drawing:

FIG. 1 shows a proximal femur in longitudinal section,

FIG. 2 shows a proximal femur with a head which has been prepared for implantation of a hip-joint endoprosthesis according to the invention,

FIGS. 3a, b show a first illustrative embodiment of a hip-joint endoprosthesis according to the invention,

FIG. 4 shows a second illustrative embodiment of a stem of an endoprosthesis according to the invention,

FIG. 5 shows a second illustrative embodiment of a hip-joint endoprosthesis according to the invention in cross section, and

FIGS. 6a, b show a model illustrating the force introduction according to the invention into the femoral neck.

### DESCRIPTION OF THE ILLUSTRATIVE EMBODIMENTS

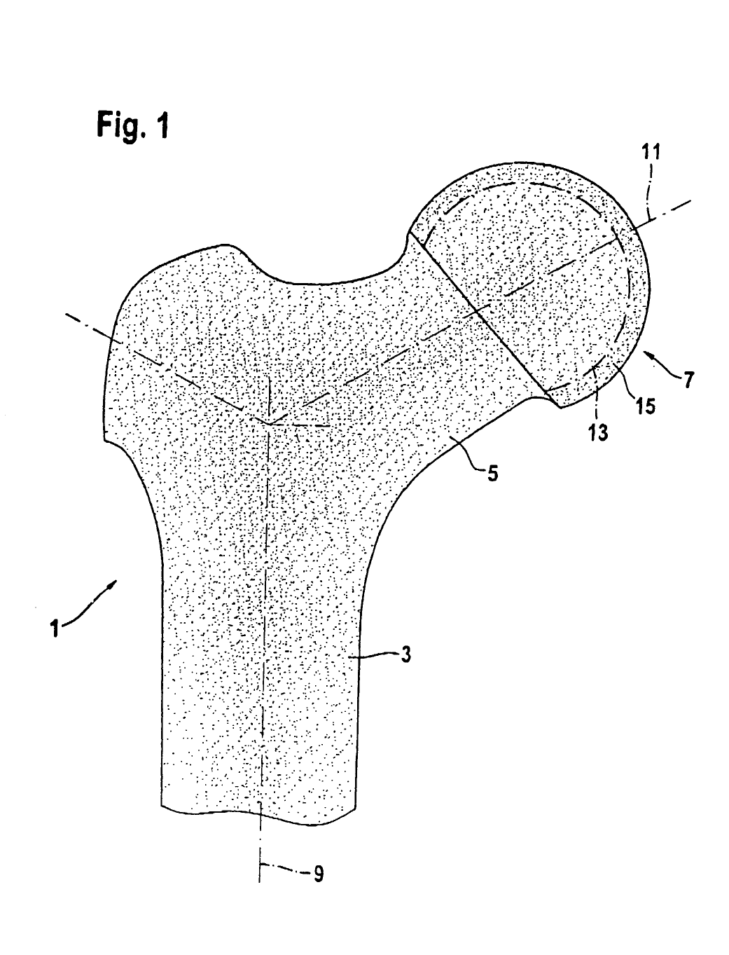

FIG. 1 shows a proximal femur 1 in longitudinal section. A femoral neck 5 with a head 7 adjoins a femoral bone 3 of the femur 1. A longitudinal axis 9 of the femoral bone 3 and a longitudinal axis 11 of the femoral neck 5 enclose an angle of approximately 120°. A broken line 13 indicates the boundary between cortical substance 15 and the spongy substance (not shown) in the area of the head 7.

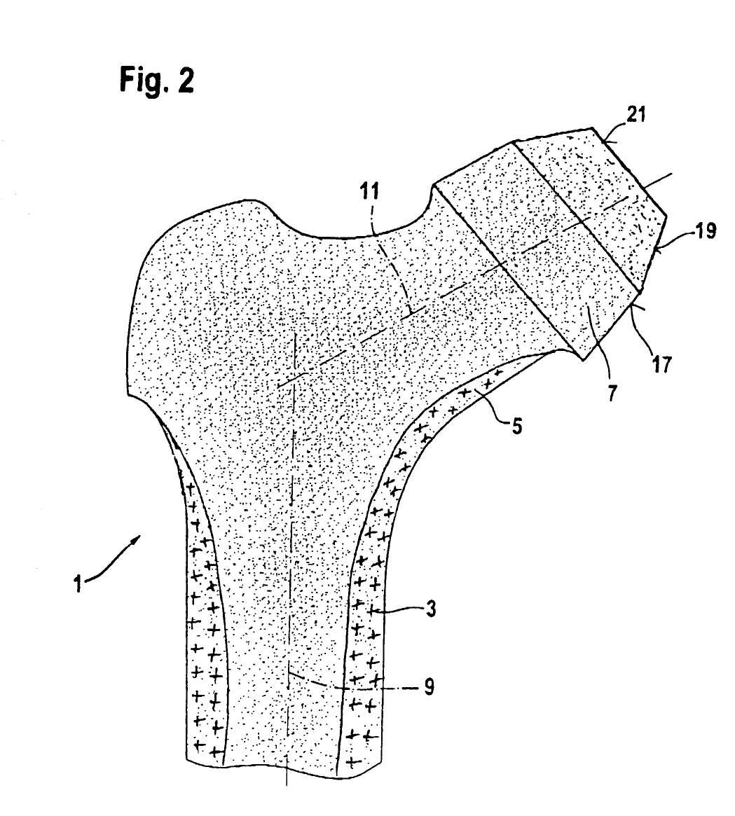

If the hip joint consisting of head 7 and a socket (not shown) has to be replaced, the head of the femur is prepared in accordance with FIG. 2. It will be seen from FIG. 2 that the head 7 has been cut away so that it assumes the shape of a double truncated cone. A first truncated cone is designated by reference number 17, while reference number 19 designates an adjoining second truncated cone. Contiguous with the second truncated cone 18 there is an end face 21. The rotationally symmetrical double truncated cone 17, 19 can be produced relatively simply and with good precision even under operating conditions. A sufficient amount of material is cut away so that the cortical substance 15 (see FIG. 1) of the head 7 is completely removed. In other words, the head which has been prepared in accordance with FIG. 2 consists exclusively of spongy substance. The number of truncated cones 17, 19 and their cone angle is not limited to the illustrative embodiment according to the invention.

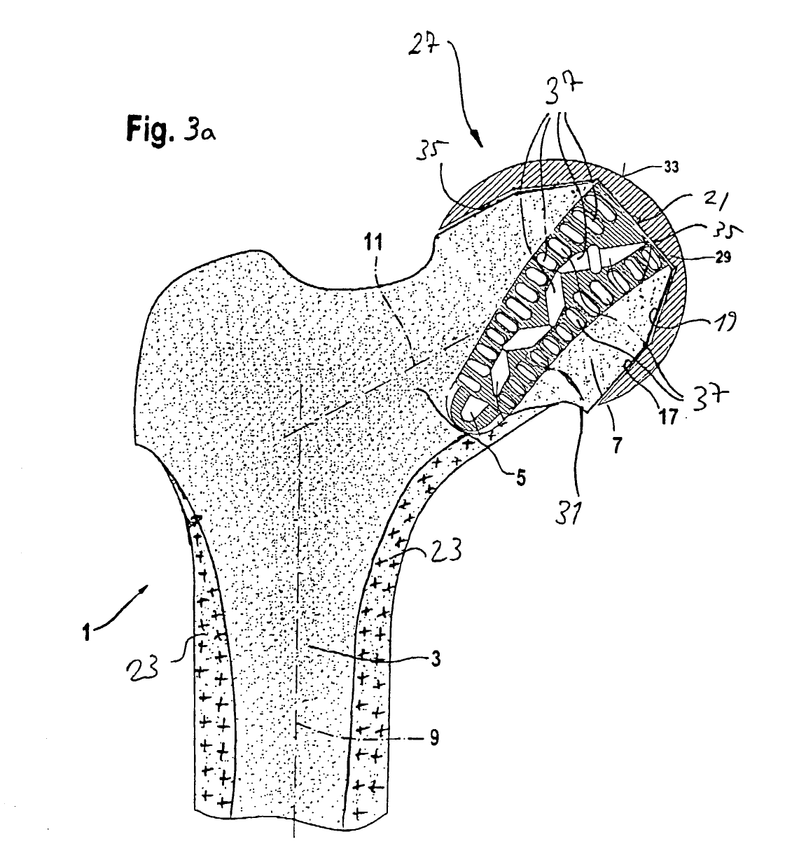

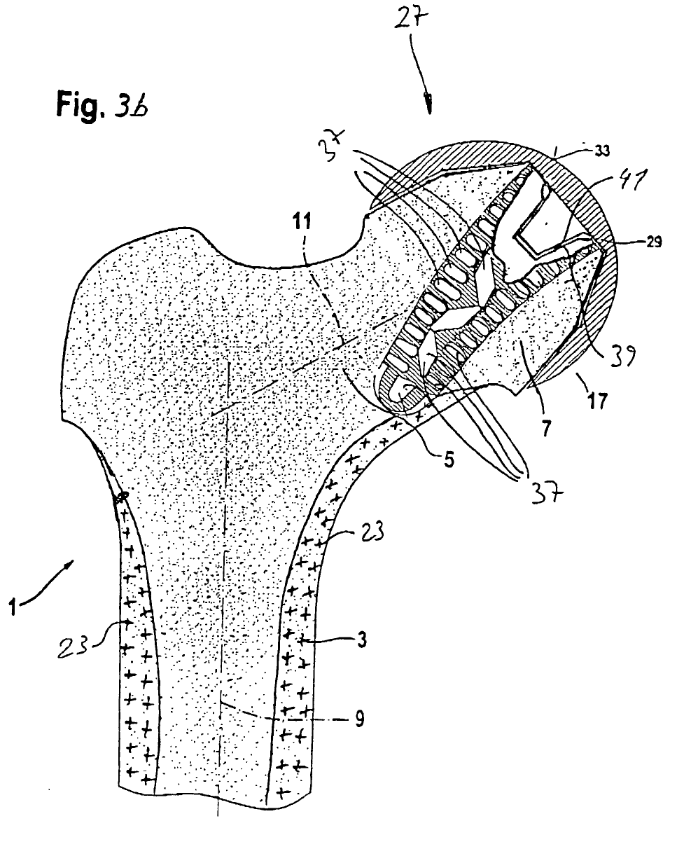

In FIG. 3a, a first illustrative embodiment of a hip-joint endoprosthesis 27 according to the invention is shown in position on the head 7 which has been prepared in accordance with FIG. 2. The hip-joint endoprosthesis 27 consists of a cap 29 and of a stem 31. The cap 29 has a spherical outer face 33, and an inner face 35 which is configured in a double truncated cone shape. Comparing FIGS. 2 and 3a, it will be clear that the inner face 35 of the cap 29 has the same shape as the prepared head 7. A frictional and form-fit connection is thus obtained between the cap 29 and the head 7 when the hip-joint endoprosthesis 27 is implanted into the femoral neck 5.

The stem 31 has a lattice-like structure with differently arranged and differently oriented openings 37. For purposes of clarity, not all the openings are provided with reference numbers in FIG. 3. As regards the shape, orientation and size of the openings 37, it is to be ensured that the spongy substance can grow as easily as possible into the openings and that a large surface area of the stem 31 is available. The following principle holds: the larger the surface area of the stem 31, the better the connection between spongy substance and stem 31 when the spongy substance has grown in.

As regards the design of the stem 31, it must also be ensured that the stem has a certain elasticity in order, in the first instance, to avoid local peak loads acting on the resection area, by some of the forces which act on the cap being taken up by the stem 31 and the forces which act on the resection surfaces of the femoral head 7 thus being reduced. The stem 31 thus has the important function of a shock absorber, the cushioning being effected by the friction between stem and spongy substance and by the flow of viscous fluid around the lattice-like structure of the stem, which fluid is located in the cavities of the spongy substance and flows into the implanted stem structure. Over the course of time of the incorporation of the cap, the stem gradually becomes superfluous, i.e. the stem 31 takes over at least part of the function of the cartilaginous tissue in the natural joint. If the cap 29 could be coated with a material having a sufficient cushioning action, it might be possible to dispense with the stem 31. Secondly, growth of spongy substance is intended to be induced by the introduction of forces. An expansion of 0.2%, in particular in the range of 0.05% to 0.3%, is well-suited to promote this effect (see: Kaspar, Seidl, Ignatius, Neidlinger-Wilke and Claes in Der Orthopäde, 2000, 29, pages 85-90, and Frots, H. M. (1992): Perspectives: bone's mechanical usage windows, Bone Miner 19, pages 257-271). In addition, the stem 31 is intended to have a cushioning which corresponds as far as possible to the function of the natural spongy substance. The modulus of elasticity of the stem 31 must be greater than the modulus of elasticity of the spongy substance in the area of the resection surfaces of the femoral head 7.

From FIG. 3a (as a possible illustrative embodiment), it will also be seen that the stem 31 bears on cortical substance 23 in the area of the femoral neck 5 or at least reaches into the immediate vicinity thereof. The forces remaining in the stem 31 are thus carried off into the cortical substance 23. It will also be seen from FIG. 3a that the longitudinal axis of the stem 31 is not parallel or coaxial to the longitudinal axis 11 of the femoral neck 5. The longitudinal axis of the stem 31 instead extends in the direction of the main loading. This direction can be visualized, for example in a cross section through a specimen, by the orientation of the trabeculae (not shown). With this orientation of the stem 31, the physiological loading is disturbed to the slightest possible extent even after implantation of the hip-joint endoprosthesis 27 into the femoral neck 5.

The socket interacting with the cap 29 is not shown in FIG. 3. This socket can be anchored in the hip bone in accordance with the prior art.

In FIG. 3b, the stem 31 is shown in partially cut-away view. The connection between stem 31 and cap 29 can therefore be seen here. For various reasons, it has in fact proven advantageous for cap 29 and stem 31 to be designed as two pieces. The optimum choice of material, production and implantation are important reasons here in favor of the two-part design.

A conical recess 39 is provided at one end of the stem 31. The cone angle and diameter (neither of which are indicated in FIG. 3b) of the recess 39 are dimensioned in such a way that they can safely transmit the forces introduced from the cap 29 into the femoral bone, likewise not shown. The associated cap 29 is likewise shown in section in FIG. 3b. The cap 29 has a peg 41 which, together with the recess 39, permits a clamp connection between cap 29 and stem 31. FIG. 3b shows the cap 29 and stem 31 when joined together. The modular construction of the second illustrative embodiment permits separate production and optimization of stem 31 and cap 29 in terms of material, production method, surface treatment and surface coating. The transition between stem 31 and inner face 35 can be rounded, as is the case in FIG. 3b, in order to reduce the notch effect.

It is also possible to design the stem 31 in the form of a framework, this framework consisting of rods and nodes which are oriented in different spatial directions (not shown). In this case, it is advisable, as in any framework, to provide rods in the direction of the impinging forces. The stability of the framework is thus optimum while having the lowest weight.

The following dimensions have proven advantageous:

| | Diameter of the | Diameter of the |
|---|---|---|
| Location | struts | openings |
| proximal third | ca. 0.15 mm | ca. 1 mm |
| middle third | ca. 0.2 mm | ca. 2 mm |
| distal third | ca. 0.25 mm | ca. 3 mm |

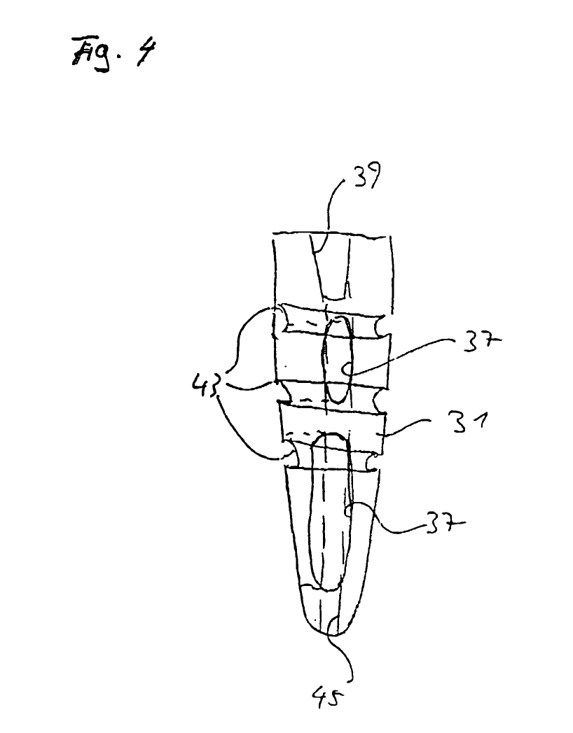

FIG. 4 shows a second illustrative embodiment of a stem 31. In addition to the openings 37, this illustrative embodiment also has circumferential grooves 43 and a longitudinal bore 45. A cap 29 (not shown) according to FIG. 3 can be fitted into the recess 39.

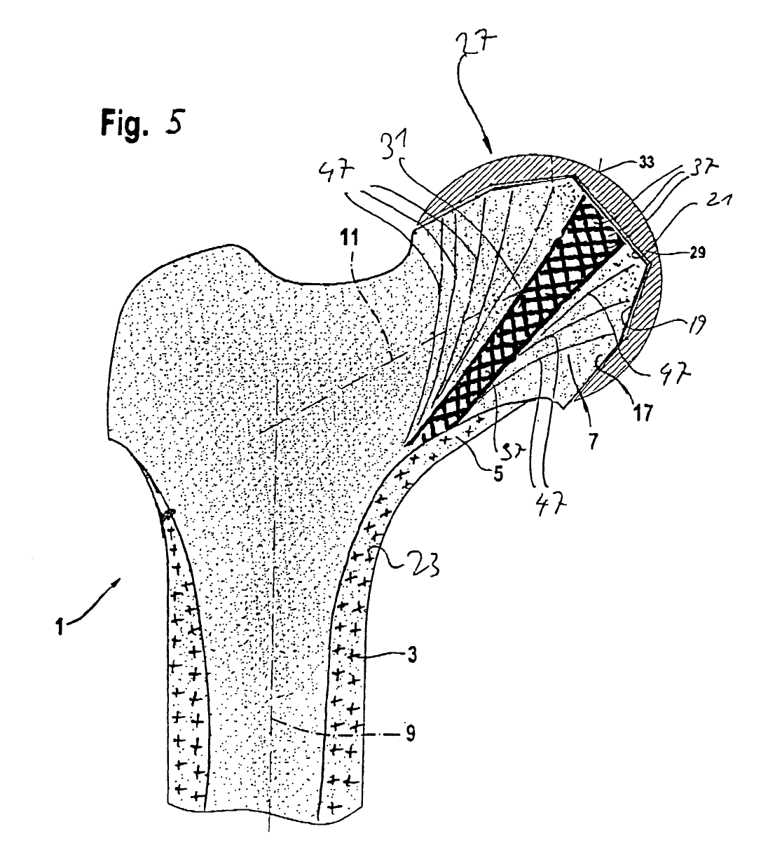

FIG. 5 shows a further illustrative embodiment of a hip-joint endoprosthesis 27 according to the invention in cross section. The cap 29 is of almost identical design to the cap 29 described with reference to FIGS. 3a and 3b. The main difference from the first illustrative embodiment according to FIG. 3 lies in the stem 31. The stem 31 is configured such that it further improves the introduction of force into the spongy substance of the head 7. It must first be stated that the spongy substance consists of trabeculae (not shown) which are oriented along the principal stress trajectories 47. In a healthy hip joint, an impulse is received by the trabeculae and some of this is introduced via the adjacent trabecular structure (not shown) into the cortical substance 23. In this way, the mechanical stressing in the spongy substance of the femoral neck 5 is reduced. The stem 31 according to the invention in FIG. 5 now imitates the structure of the spongy substance in the area of the principal stress trajectories 47. It is funnel-shaped at the proximal end, but narrows in the distal direction and finally contacts the medial cortical substance 23. It is constructed in the form of a lattice, e.g. as a two-dimensional or three-dimensional tissue, with openings 37 whose diameter corresponds approximately to the pore size of the spongy substance. Openings 37 with a diameter of between 1 mm and 4 mm have proven advantageous.

The stem 41 is of rotationally symmetrical design and ends flush with the end face 21 of the head 7. At its other end, it is beveled and ideally rests on the cortical substance 23.

Between the cap 29 and the stem 41 there is no connection via a recess 39 and a peg 41. The force is instead transmitted from the cap 29 to the stem 41 by the fact the that surface of the cap 29 corresponding to the end face 21 lies on the stem 41 without clearance, and pressure forces can thus be transmitted from the cap 29 to the stem 41. The construction of the cap is tailored to the stem in such a way that the system is statically neutral, but dynamically active.

In this illustrative embodiment too, the longitudinal axis of the stem 41 extends approximately in the direction of the principal trajectories 47, i.e. the principal direction of attack of the forces acting on the femoral neck 5.

Because of the openworked, lattice-like structure of the stem 41, the latter is particularly soft and flexible, so that there is a very gentle introduction of force from the stem 41 into the spongy substance surrounding it. In addition, because of the very large number of openings 37 in the stem 41, the spongy substance is able to grow very satisfactorily into the stem 41 and connect intimately with the latter.

However, the stem 41 is slightly more rigid than the spongy substance surrounding it, specifically to such an extent that the shocks from the cap 29 are taken up by the stem 41 and passed on to the cortical substance 23 to such a degree that the spongy substance in the area of the femoral neck is not damaged.

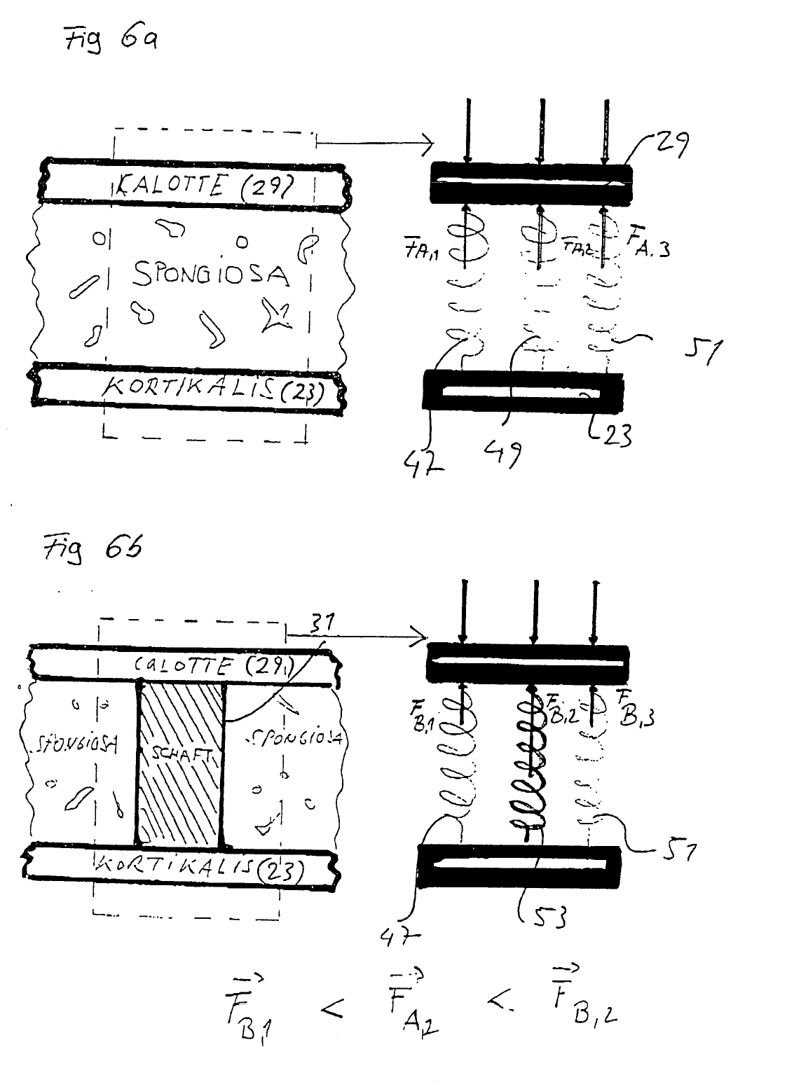

FIGS. 6a and 6b are intended to illustrate the operating principle of the hip-joint endoprosthesis according to the invention on the basis of a model. Here, the model consists of three elements, namely the cap 29, the cortical substance 23 of the femoral neck 5, and the spongy substance arranged between these. If the cap 29 and the cortical substance 23 are considered here in simplified terms as rigid bodies, then the entire elasticity and cushioning is afforded by the spongy substance. In FIG. 6a, the elastic property of the spongy substance is indicated by three springs 47, 49 and 51 of identical spring rate. The forces introduced via the cap 29 generate, in the springs 47, 49 and 51, identical counterforces F_{A,1}=F_{A,2}=F_{A,3}.

In FIG. 6b, the model is varied slightly in that a stem 31 is provided between cap 29 and cortical substance 23 in addition to the spongy substance, the rigidity of this stem 31 being slightly greater than that of the spongy substance surrounding it. Transposed to the model with three springs, which is shown on the right-hand side of FIG. 6b, this means that the middle spring 53 has a greater spring rate than the springs 47 and 51 which represent the spongy substance surrounding the stem 31. This means that the forces transmitted by the springs 47, 51 and 53 are not equal. Instead, the force F_{B,1} transmitted by the spring 47 and the force F_{B,3} transmitted by the spring 51 are smaller than the force F_{B,2} transmitted by the spring 53. Since, if we assume that a constant external force acts on the cap 29, the sum of F_{B,1}+F_{B,2}+F_{B,3} is nevertheless identical to the forces F_{A,1}+F_{A,2}+F_{A,3} (see FIG. 6a), this can only mean that the stem 31 relieves the load on the spongy substance surrounding it, since the forces F_{B,1} and F_{B,3} are reduced by the presence of the stem 31 with a greater rigidity than the spongy substance surrounding it.

Because of the lattice-like structure of the stem 31, it is possible to dispense with the use of cement, which fact brings considerable advantages in terms of the strength and stability of the anchoring of the hip-joint endoprosthesis in the femur. The advantages of cementless anchoring are familiar enough. However, it has not hitherto been possible to secure a surface replacement for the proximal femur permanently in the neck 5 without using cement.

1. A hip-joint endoprosthesis as a surface replacement for the proximal femur, with a cap (29), said cap (29) having an outer face (33), which interacts with a socket, and an inner face (35), and with a stem (31), characterized in that the stem (31) can be connected to the cap (29), and in that the stem (31) has a lattice-like structure.

2. The hip-joint endoprosthesis as claimed in claim 1, characterized in that the rigidity of the stem (31), in particular in the longitudinal direction of the stem (31), is greater than the spongy substance surrounding the stem (31).

3. The hip-joint endoprosthesis as claimed in claim 1 or 2, characterized in that the rigidity of the stem (31), in particular in the longitudinal direction of the stem (31), is less than the cortical substance of the femoral neck.

4. The hip-joint endoprosthesis as claimed in one of the preceding claims, characterized in that the lattice-like structure of the stem (31) is composed of openings (37) in the stem (31), meshes or rods and bridges.

5. The hip-joint endoprosthesis as claimed in one of the preceding claims, characterized in that the lattice-like structure has thickened areas and/or depressions.

6. The hip-joint endoprosthesis as claimed in one of the preceding claims, characterized in that the longitudinal axis of the stem (31) extends in the direction of the greatest loading of the femoral neck (5).

7. The hip-joint endoprosthesis as claimed in one of the preceding claims, characterized in that the first third of the stem (31) is connected to the cap in a flexurally rigid manner, and in that the first third of the stem (31) in the longitudinal direction has a rigidity lying within the limits of claims 2 and 3.

8. The hip-joint endoprosthesis as claimed in one of the preceding claims, characterized in that the second third of the stem (31) is flexible, and in that the second third of the stem (31) in the longitudinal direction has a rigidity lying within the limits of claims 2 and 3.

9. The hip-joint endoprosthesis as claimed in one of the preceding claims, characterized in that the third third of the stem (31) is flexible, and in that the third third of the stem (31) in the longitudinal direction has a rigidity greater than the rigidity of the cortical substance (23) of the femoral neck (5).

10. The hip-joint endoprosthesis as claimed in one of the preceding claims, characterized in that the rods of the stem (31) in the proximal third have a diameter of ca. 0.15 mm and a spacing of ca. 1 mm from one another, in the middle third a diameter of ca. 0.2 mm and a spacing of ca. 2 mm from one another, and, in the distal third, a diameter of ca. 0.25 mm and a spacing of ca. 3 mm from one another.

11. The hip-joint endoprosthesis as claimed in one of the preceding claims, characterized in that, in the implanted state, the stem (31) has an expansion of 0.2%, in particular of 0.05% to 0.3%, under the loads acting on the surface.

12. The hip-joint endoprosthesis as claimed in one of the preceding claims, characterized in that the surface of the stem (31) is porous or periplasmal.

13. The hip-joint endoprosthesis as claimed in one of the preceding claims, characterized in that the surface of the stem (31) is hydroxyapatite.

14. The hip-joint endoprosthesis as claimed in one of the preceding claims, characterized in that the inner face (35) corresponds to the surface of a truncated cone (17, 19, 21), in particular of a double truncated cone.

15. The hip-joint endoprosthesis as claimed in one of the preceding claims, characterized in that the diameter or diameters of the inner face (35) are dimensioned such that they are smaller than the cortical substance (15) of the head (7) of the femur (1).

16. The hip-joint endoprosthesis as claimed in one of the preceding claims, characterized in that the cap (29) and the stem (31) are connected to one another by a clamp connection.

17. The hip-joint endoprosthesis as claimed in claim 16, characterized in that the stem (31) has a conical recess (39), and in that the cap (29) has a peg (41), and in that the recess (39) and the peg (41) can be connected by clamping.

18. The hip-joint endoprosthesis as claimed in claim 17, characterized in that the cross section of the conical recess (39) and of the peg (41) is round, polygonal, rectangular or square.

19. The hip-joint endoprosthesis as claimed in one of the preceding claims, characterized in that the cap (29) and the stem (31) are designed in one piece.

20. The hip-joint endoprosthesis as claimed in one of the preceding claims, characterized in that the cap (29) and the stem (31) are designed in two pieces, and in that the cap (29) rests on the stem (31) in the implanted state.

21. The hip-joint endoprosthesis as claimed in one of the preceding claims, characterized in that the outer face (33) of the cap (29) is provided with a coating which provides a cushioning action, lessens wear and reduces friction.

22. The hip-joint endoprosthesis as claimed in one of the preceding claims, characterized in that the hip-joint endoprosthesis is made at least partially from titanium, in particular TiAl6V4, a steel alloy or CoCr, by casting and/or forging, of sintered metal or of a porous ceramic or of biodegradable material.

23. The hip-joint endoprosthesis as claimed in one of the preceding claims, characterized in that the construction of the cap is matched to the stem in such a way that the system is statically neutral, but dynamically active.
